(19) United States
(12) Patent Application Publication
(10) Pub. No.: US 2012/0071325 A1
(43) Pub. Date: Mar. 22, 2012

(54) USE OF AMIDE COMPOUND FOR PROMOTING PLANT ROOT GROWTH

(76) Inventor: Shinya Nishimura, Yokohama-shi (JP)

(21) Appl. No.: 13/319,385

(22) PCT Filed: May 12, 2010
(86) PCT No.: PCT/JP2010/058401

§ 371 Date: Dec. 2, 2011

(30) Foreign Application Priority Data
May 13, 2009 (JP) 2009-116871

(51) Int. Cl. A01N 43/56; A01P 21/00; C07D 401/04

(52) U.S. Cl. 504/253; 546/275.4; 546/276.1

(57) ABSTRACT

The present invention relates to use of amide compounds for promoting root growth of plants.

### TECHNICAL FIELD

The present invention relates to use of amide compounds for promoting root growth of plants.

### BACKGROUND ART

Certain amide compounds (see, for example, WO2007-043677) have been known as compounds which control arthropods.

### DISCLOSURE OF INVENTION

An object of the present invention is to provide a root growth promoter and a method of promoting root growth of plants, having excellent plant root growth-promoting effect.

The present invention is based on specific amide compounds having activity of promoting root growth of plants.

The present invention has the following constitutions:

[1] A root growth promoter comprising, as an active ingredient, a compound of formula (1):

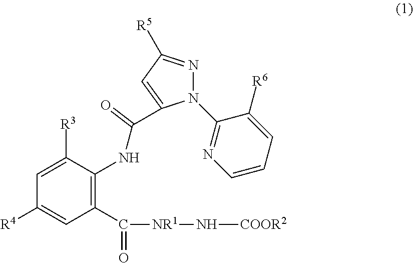

wherein R¹ represents a C1-C6 alkyl group optionally substituted with (a) halogen atom(s); R² represents a C1-C6 alkyl group optionally substituted with (a) halogen atom(s), a C3-C6 alkoxyalkyl group optionally substituted with (a) halogen atom(s), a C3-C6 alkenyl group optionally substituted with (a) halogen atom(s), or a C3-C6 alkynyl group optionally substituted with (a) halogen atom(s); R³ represents a halogen atom, or a C1-C6 alkyl group optionally substituted with (a) halogen atom(s); R⁴ represents a hydrogen atom, a halogen atom, a cyano group, or a C1-C6 alkyl group optionally substituted with (a) halogen atom(s); R⁵ represents a hydrogen atom, a halogen atom, a cyano group, a C1-C6 alkyl group optionally substituted with (a) halogen atom(s), a C1-C6 alkoxy group optionally substituted with (a) halogen atom(s), a C1-C6 alkylthio group optionally substituted with (a) halogen atom(s), a C1-C6 alkylsulfinyl group optionally substituted with (a) halogen atom(s), or a C1-C6 alkylsulfonyl group optionally substituted with (a) halogen atom(s); and R⁶ represents a halogen atom, or a C1-C6 alkyl group optionally substituted with (a) halogen atom(s);
[2] A method of promoting root growth of plants, which comprises applying an effective amount of a compound of formula (1)

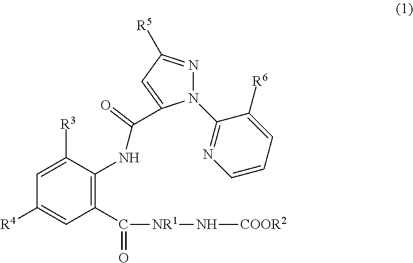

wherein R¹ represents a C1-C6 alkyl group optionally substituted with (a) halogen atom(s); R² represents a C1-C6 alkyl group optionally substituted with (a) halogen atom(s), a C3-C6 alkoxyalkyl group optionally substituted with (a) halogen atom(s), a C3-C6 alkenyl group optionally substituted with (a) halogen atom(s), or a C3-C6 alkynyl group optionally substituted with (a) halogen atom(s); R³ represents a halogen atom, or a C1-C6 alkyl group optionally substituted with (a) halogen atom(s); R⁴ represents a hydrogen atom, a halogen atom, a cyano group, or a C1-C6 alkyl group optionally substituted with (a) halogen atom(s); R⁵ represents a hydrogen atom, a halogen atom, a cyano group, a C1-C6 alkyl group optionally substituted with (a) halogen atom(s), a C1-C6 alkoxy group optionally substituted with (a) halogen atom(s), a C1-C6 alkylthio group optionally substituted with (a) halogen atom(s), a C1-C6 alkylsulfinyl group optionally substituted with (a) halogen atom(s), or a C1-C6 alkylsulfonyl group optionally substituted with (a) halogen atom(s); and R⁶ represents a halogen atom, or a C1-C6 alkyl group optionally substituted with (a) halogen atom(s);
to a plant or growing site of plant;
[3] The method of promoting root growth of plants according to [2], wherein the plant is seed or seedling;
[4] The method of promoting root growth of plants according to [2], wherein the growing site of plant is soil before or after sowing plant;
[5] The method of promoting root growth of plants according to [2], wherein the growing site of plant is culture solution; and
[6] Use of the compound of formula (1) of [1] for promoting root growth of plants.

The root growth promoter of the present invention can promote root growth of plants.

### MODE FOR CARRYING OUT THE INVENTION

The root growth promoter of the present invention comprises, as an active ingredient, a compound of formula (1):

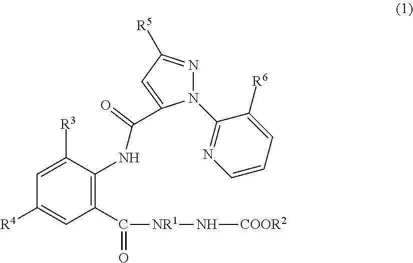

wherein R¹ represents a C1-C6 alkyl group optionally substituted with (a) halogen atom(s); R² represents a C1-C6 alkyl group optionally substituted with (a) halogen atom(s), a C3-C6 alkoxyalkyl group optionally substituted with (a) halogen atom(s), a C3-C6 alkenyl group optionally substituted with (a) halogen atom(s), or a C3-C6 alkynyl group optionally substituted with (a) halogen atom(s); R³ represents a halogen atom, or a C1-C6 alkyl group optionally substituted with (a) halogen atom(s); R⁴ represents a hydrogen atom, a halogen atom, a cyano group, or a C1-C6 alkyl group optionally substituted with (a) halogen atom(s); R⁵ represents a hydrogen atom, a halogen atom, a cyano group, a C1-C6 alkyl group optionally substituted with (a) halogen atom(s), a C1-C6 alkoxy group optionally substituted with (a) halogen atom(s), a C1-C6 alkylthio group optionally substituted with (a) halogen atom(s), a C1-C6 alkylsulfinyl group optionally substituted with (a) halogen atom(s), or a C1-C6 alkylsulfonyl group optionally substituted with (a) halogen atom(s); and R⁶ represents a halogen atom, or a C1-C6 alkyl group optionally substituted with (a) halogen atom(s) (hereinafter, referred to as the present compound in some cases).

Examples of members represented by R¹ to R⁶ in the formula (1) include the following members.

Examples of “halogen atom” include a fluorine atom, chlorine atom, bromine atom and iodine atom.

Examples of “C1-C6 alkyl group optionally substituted with (a) halogen atom(s)” include a methyl group, trifluoromethyl group, trichloromethyl group, chloromethyl group, dichloromethyl group, fluoromethyl group, difluoromethyl group, ethyl group, pentafluoroethyl group, 2,2,2-trifluoroethyl group, 2,2,2-trichloroethyl group, propyl group, isopropyl group, heptafluoroisopropyl group, butyl group, isobutyl group, sec-butyl group, tert-butyl group, pentyl group and hexyl group.

Examples of “C3-C6 alkoxyalkyl group optionally substituted with (a) halogen atom(s)” include a 2-methoxyethyl group, 2-ethoxyethyl group and 2-isopropyloxyethyl group.

Examples of “C3-C6 alkenyl group optionally substituted with (a) halogen atom(s)” include a 2-propenyl group, 3-chloro-2-propenyl group, 2-chloro-2-propenyl group, 3,3-dichloro-2-propenyl group, 2-butenyl group, 3-butenyl group, 2-methyl-2-propenyl group, 3-methyl-2-butenyl group, 2-pentenyl group and 2-hexenyl group.

Examples of “C3-C6 alkynyl group optionally substituted with (a) halogen atom(s)” include a 2-propynyl group, 3-chloro-2-propynyl group, 3-bromo-2-propynyl group, 2-butynyl group and 3-butynyl group.

Examples of “C1-C6 alkoxy group optionally substituted with (a) halogen atom(s)” include a methoxy group, ethoxy group, 2,2,2-trifluoroethoxy group, propoxy group, isopropyloxy group, butoxy group, isobutyloxy group, sec-butoxy group and tert-butoxy group.

Examples of “C1-C6 alkylthio group optionally substituted with (a) halogen atom(s)” include a methylthio group, trifluoromethylthio group, ethylthio group, propylthio group, isopropylthio group, butylthio group, isobutylthio group, sec-butylthio group, tert-butylthio group, pentylthio group and hexylthio group.

Examples of “C1-C6 alkylsulfinyl group optionally substituted with (a) halogen atom(s)” include a methylsulfinyl group, trifluoromethylsulfinyl group, ethylsulfinyl group, propylsulfinyl group, isopropylsulfinyl group, butylsulfinyl group, isobutylsulfinyl group, sec-butylsulfinyl group, tert-butylsulfinyl group, pentylsulfinyl group and hexylsulfinyl group.

Examples of “C1-C6 alkylsulfonyl group optionally substituted with (a) halogen atom(s)” include a methylsulfonyl group, trifluoromethylsulfonyl group, ethylsulfonyl group, propylsulfonyl group, isopropylsulfonyl group, butylsulfonyl group, isobutylsulfonyl group, sec-butylsulfonyl group, tert-butylsulfonyl group, pentylsulfonyl group and hexylsulfonyl group.

Examples of the present compound include the following compounds.

A compound of the formula (1) in which R¹ is a methyl group, ethyl group or isopropyl group, R² is a methyl group or ethyl group, R³ is a halogen atom or methyl group, R⁴ is a halogen atom or cyano group, R⁵ is a halogen atom or trifluoromethyl group and R⁶ is a halogen atom.

A compound of the formula (1) in which R¹ is a methyl group, R² is a methyl group, R³ is a chlorine atom, bromine atom or methyl group, R⁴ is a chlorine atom, bromine atom or cyano group, R⁵ is a chlorine atom, bromine atom or trifluoromethyl group and R⁶ is a chlorine atom.

A compound of the formula (1) in which R¹ is an ethyl group, R² is a methyl group, R³ is a chlorine atom, bromine atom or methyl group, R⁴ is a chlorine atom, bromine atom or cyano group, R⁵ is a chlorine atom, bromine atom or trifluoromethyl group and R⁶ is a chlorine atom.

Specific examples of the present compound include compounds 1 to 26 in which R¹ to R⁶ of the formula (1) are one of the combinations of members shown in Table 1.

TABLE 1

| Compound No. | R¹ | R² | R³ | R⁴ | R⁵ | R⁶ |
|---|---|---|---|---|---|---|
| 1 | CH₃CH₂ | CH₃ | Br | Br | Br | Cl |
| 2 | CH₃ | CH₃ | CH₃ | Cl | Br | Cl |
| 3 | CH₃ | CH₃ | Br | Br | Br | Cl |
| 4 | (CH₃)₂CH | CH₃ | Br | Br | Br | Cl |
| 5 | CH₃ | CH₃ | CH₃ | Cl | CF₃ | Cl |
| 6 | CH₃ | CH₃ | CH₃ | CN | Br | Cl |
| 7 | CH₃ | CH₃ | Cl | Cl | Br | Cl |
| 8 | CH₃ | CH₃CH₂ | Cl | Cl | Br | Cl |
| 9 | CH₃CH₂ | CH₃ | Cl | Cl | Br | Cl |
| 10 | CH₃(CH₂)₂ | CH₃ | Br | Br | Br | Cl |
| 11 | CH₃CH₂ | CH₃ | CH₃ | Cl | Br | Cl |
| 12 | CH₃CH₂ | CH₃ | CH₃ | CN | Br | Cl |
| 13 | CH₃CH₂ | CH₃ | Br | Br | Cl | Cl |
| 14 | CH₃CH₂ | CH₃ | Cl | Cl | Cl | Cl |
| 15 | CH₃CH₂ | CH₃ | CH₃ | Cl | Cl | Cl |
| 16 | CH₃CH₂ | CH₃ | CH₃ | CN | Cl | Cl |
| 17 | CH₃CH₂ | CH₃ | Br | Br | CF₃ | Cl |
| 18 | CH₃CH₂ | CH₃ | Cl | Cl | CF₃ | Cl |
| 19 | CH₃CH₂ | CH₃ | CH₃ | Cl | CF₃ | Cl |
| 20 | CH₃CH₂ | CH₃ | CH₃ | CN | CF₃ | Cl |
| 21 | CH₃ | CH₃ | Br | Br | CF₃ | Cl |
| 22 | CH₃ | CH₃ | Br | Br | Cl | Cl |
| 23 | CH₃ | CH₃ | Cl | Cl | Cl | Cl |
| 24 | CH₃ | CH₃ | CH₃ | Cl | Cl | Cl |
| 25 | CH₃ | CH₃ | CH₃ | CN | Cl | Cl |
| 26 | CH₃ | CH₃ | Cl | Cl | CF₃ | Cl |

When the present compound has at least one acidic group, the compound may be a salt with a base. Examples of the salt thereof include metal salts such as alkali metal salts and alkali earth metal salts (for example, salts of sodium, potassium or magnesium); salts with ammonia; and salts with organic amines such as morpholine, piperidine, pyrrolidine, mono lower alkylamine, di lower alkylamine, tri lower alkylamine, monohydroxy lower alkylamine, dihydroxy lower alkylamine and trihydroxy lower alkylamine.

The present compound may have isomers such as tautomers and stereoisomers including optical isomers based on an asymmetric carbon atoms, and any isomer can be contained and used solely or in a mixture of any isomer ratio in the present invention.

The present compounds are compounds described in WO2007-043677. These compounds can be produced, for example, by the method described in the publication.

As the root growth promoter of the present invention, the present compound may be used as it is, but is typically prepared into a formulation such as emulsifiable concentrate, liquid agent, microemulsion, flowable agent, oil agent, wettable powder, granulated wettable powder, water soluble powder, dust formulation, granule, microgranule, seed-coating agent, seed-soaking agent, smoking agent, tablet, microcapsule, spray, aerosol, carbon dioxide gas preparaton, EW agent, trunk injection and trunk-coating agent by mixing one or more kinds, preferably one to three kinds of the present compounds with an inert carrier and adding as needed surfactant and other adjuvants for the formulation to the mixture.

Examples of the solid carrier (dilution agent, extending agent) which can be used in the preparations include fine powders or granules such as plant powders (for example, soybean flour, tobacco flour, wheat flour, wood flour and so on), mineral powders (for example, clays such as kaolin clay, Fubasami clay, bentonite and acid clay, talcs such as talc powder and agalmatolite powder, silicas such as diatomaceous earth and mica powder, and so on), synthetic hydrated silicon oxide, alumina, talc, ceramic, other inorganic minerals (sericite, quartz, sulfur, active carbon, calcium carbonate, hydrated silica and so on) and chemical fertilizers (ammonium sulfate, ammonium phosphate, ammonium nitrate, urea, ammonium chloride). One or more (preferably, one or more and three or less) of these solid carriers may be mixed at suitable proportion and used.

Examples of the liquid carrier include water, alcohols (for example, methyl alcohol, ethyl alcohol, n-propyl alcohol, isopropyl alcohol, butyl alcohol, hexyl alcohol, benzyl alcohol, ethylene glycol, propylene glycol, phenoxyethanol and so on), ketones (for example, acetone, methyl ethyl ketone, methyl isobutyl ketone, cyclohexanone and so on), ethers (for example, diisopropyl ether, 1,4-dioxane, tetrahydrofuran, ethylene glycol monomethyl ether, ethylene glycol dimethyl ether, diethylene glycol monomethyl ether, propylene glycol monomethyl ether, dipropylene glycol monomethyl ether, 3-methoxy-3-methyl-1-butanol and so on), aliphatic hydrocarbons (for example, hexane, cyclohexane, kerosene, lamp oil, fuel oil, machine oil and so on), aromatic hydrocarbons (for example, toluene, xylene, ethylbenzene, dodecylbenzene, phenylxylylethane, solvent naphtha, methylnaphthalene and so on), halogenated hydrocarbons (for example, dichloromethane, trichloroethane, chloroform, carbon tetrachloride and so on), acid amides (for example, N,N-dimethylformamide, N,N-dimethylacetamide, N-methylpyrrolidone, N-octylpyrrolidone and so on), esters (for example, butyl lactate, ethyl acetate, butyl acetate, isopropyl myristate, ethyl oleate, diisopropyl adipate, diisobutyl adipate, propylene glycol monomethyl ether acetate, fatty acid glycerin ester, γ-butyrolactone and so on), nitriles (for example, acetonitrile, isobutyronitrile, propionitrile and so on), carbonates (for example, propylene carbonate and so on), and vegetable oils (for example, soybean oil, olive oil, linseed oil, coconut oil, palm oil, peanut oil, malt oil, almond oil, sesame oil, mineral oil, rosmarinic oil, geranium oil, rapeseed oil, cotton seed oil, corn oil, safflower oil, orange oil and so on). One or more (preferably, one or more and three or less) of these liquid carriers may be mixed at suitable proportion and used.

Examples of the gaseous carrier include fluorocarbon, butane gas, LPG (liquefied petroleum gas), dimethyl ether and carbon dioxide gas. These gaseous carriers can be used singly or two of them can be mixed in suitable proportion, or can be combined with a suitable liquid carrier, and used.

Examples of the surfactant include nonionic and anionic surfactants such as soaps, polyoxyethylene alkyl aryl ethers (for example, Noigen (product name, registered trademark, manufactured by Dai-Ichi Kogyo Seiyaku Co., Ltd.), EA142 (EA142(product name, manufactured by Dai-Ichi Kogyo Seiyaku Co., Ltd.)), Nonal (product name, manufactured by Toho Chemical Industry Co., Ltd.)), alkylsulfates (for example, Emal 10 (product name, registered trademark, manufactured by Kao Corporation), Emal 40 (product name, registered trademark, manufactured by Kao Corporation)), alkylbenzene sulfonates (for example, Neogen (product name, registered trademark, manufactured by Dai-Ichi Kogyo Seiyaku Co., Ltd.), Neogen T (product name, registered trademark, manufactured by Dai-Ichi Kogyo Seiyaku Co., Ltd.), Neopelex (product name, registered trademark, manufactured by Kao Corporation), polyethylene glycol ethers (for example, Nonipole 85 (product name, registered trademark, manufactured by Sanyo Chemical Industries, Ltd.), Nonipole 100 (product name, registered trademark, manufactured by Sanyo Chemical Industries, Ltd.), Nonipole 160 (product name, registered trademark, manufactured by Sanyo Chemical Industries, Ltd.)), polyoxyethylene alkyl ethers (for example, Noigen ET-135 (product name, registered trademark, manufactured by Dai-Ichi Kogyo Seiyaku Co., Ltd.)), polyoxyethylene polyoxypropylene block polymers (for example, Newpole PE-64 (product name, registered trademark, manufactured by Sanyo Chemical Industries, Ltd.)), polyhydric alcohol esters (for example, Tween 20 (product name, registered trademark, manufactured by Kao Corporation), Tween 80 (product name, registered trademark, manufactured by Kao Corporation)), alkylsulfosuccinates (for example, Sanmorin OT20 (product name, registered trademark, manufactured by Sanyo Chemical Industries, Ltd.), Newcalgen EX70 (product name, manufactured by TAKEMOTO Oil & Fat Co., Ltd.)), alkyl naphthalene sulfonates (for example, Newcalgen WG-1 (product name, manufactured by TAKEMOTO Oil & Fat Co., Ltd.), and alkenyl sulfonates (for example, Sorpole 5115 (product name, registered trademark, manufactured by Toho Chemical Industry Co., Ltd.)). One or more (preferably, one or more and three or less) of these surfactants can be mixed in suitable proportion and used.

Examples of the other additives include casein, gelatin, saccharides (starch, xanthan gum, gum arabic, cellulose derivatives, alginic acid and so on), lignin derivatives, bentonite, synthetic water-soluble polymers (polyvinyl alcohol, polyvinyl pyrrolidone, polyacrylic acids and so on), PAP (acidic isopropyl phosphate), BHT (2,6-di-tert-butyl-4-methylphenol), and BHA (mixture of 2-tert-butyl-4-methoxyphenol and 3-tert-butyl-4-methoxyphenol).

In the root growth promoter of the present invention, the content of the present compound is typically within a range from 0.01 to 95% by weight, preferably from 0.1 to 80% by weight, and more preferably from 1 to 60% by weight. When the root growth promoter is prepared into emulsifiable concentrate, liquid agent, wettable powder, granulated wettable powder or water soluble powder, the content of the present compound is typically within a range from 1 to 90% by weight, preferably from 5 to 80% by weight, and more preferably from 5 to 60% by weight. When the root growth promoter is prepared into oil agent or dust formulation, the content of the present compound is typically within a range from 0.01 to 90% by weight, preferably from 0.1 to 50% by weight, and more preferably from 0.1 to 20% by weight. When the root growth promoter is prepared into granule, the content of the present compound is typically within a range from 0.1 to 50% by weight, preferably from 0.5 to 50% by weight, and more preferably from 1 to 20% by weight.

In the root growth promoter of the present invention, the content of a liquid carrier or a solid carrier is, for example, within a range from 1 to 90% by weight, and preferably from 1 to 70% by weight, and the content of a surfactant is, for example, within a range from 1 to 20% by weight, and preferably from 1 to 15% by weight. When the root growth promoter is prepared into liquid agent, the content of water is, for example, from 20 to 90% by weight and the content of the surfactant is from 1 to 20% by weight, and preferably from 1 to 10% by weight.

Root growth of plants can be promoted by applying an effective amount of the present compound to plants or growing sites of plants. Examples of a plant which is the object of the application include foliages, seeds, bulbs and seedlings. As used herein, the bulb means a bulb, corm, rhizoma, stem tuber, root tuber and rhizophore. In the present specification, the seedling includes cutting and sugar cane stem cutting. Examples of the growing sites of plants include soil before or after sowing plants, and a medium for water culture. Examples of the medium for water culture include water, culture solution, urethane and rock wool. As used herein, the culture solution is prepared by dissolving nutrient components required for plant growth in water so as to be adjusted to a proper concentration. The culture solution can also be used, for example, by soaking seeds or cuttings therein for their germination or rooting, or by soaking roots of plants therein or spraying it to roots to culture the plants.

Specific examples of the method of promoting root growth of plants according to the present invention include treatment of foliage of plants, such as foliage application; treatment to cultivation lands of plants, such as soil treatment; treatment of the culture solution; treatment of seeds, such as seed soaking and seed coating; treatment of seedlings, such as application, soaking and coating; and treatment of bulbs such as seed tuber.

Specific examples of the treatment of foliage of plants in the method of promoting root growth of plants according to the present invention include treatment methods of applying to surfaces of plants, such as foliage spraying and trunk spraying. Examples of the treatment method of directly absorbing to plants before transplantation include a method of soaking entire plants or roots. A formulation obtained by using a solid carrier such as a mineral powder may be adhered to the roots.

Examples of the soil treatment method in the method of promoting root growth of plants according to the present invention include spraying onto the soil, soil incorporation, and perfusion of a chemical liquid into the soil (irrigation of chemical liquid, soil injection, and dripping of chemical liquid). Examples of the place to be treated include planting hole, furrow, around a planting hole, around a furrow, entire surface of cultivation lands, the parts between the soil and the plant, area between roots, area beneath the trunk, main furrow, growing soil, seedling raising box, seedling raising tray and seedbed. Examples of the treating period include before seeding, at the time of seeding, immediately after seeding, raising period, before settled planting, at the time of settled planting, and growing period after settled planting. In the above soil treatment, active ingredients may be simultaneously applied to the plant, or a solid fertilizer such as a paste fertilizer containing active ingredients may be applied to the soil. Also active ingredients may be mixed in an irrigation liquid, and, for example, may be injected to irrigation facilities (irrigation tube, irrigation pipe, sprinkler, etc.), mixed into the flooding liquid between furrows. Alternatively, an irrigation liquid is mixed with active ingredients in advance and, for example, used for treatment by an appropriate irrigating method including the irrigating method mentioned above and the other methods such as sprinkling and flooding.

Examples of the treatment to the medium for water culture in the method of promoting root growth of plants according to the present invention include mixing into the culture solution.

The method of treating seeds or bulbs in the method of promoting root growth of plants according to the present invention is, for example, a method of treating seeds or bulbs with the root growth promoter of the present invention, and specific examples thereof include a spraying treatment in which a suspension of the root growth promoter of the present invention is atomized and sprayed over surfaces of seeds or bulbs, an smearing treatment in which a wettable powder, an emulsifiable concentrate or a flowable agent of the root growth promoter of the present invention is applied to seeds or bulbs with a small amount of water added or without dilution, an immersing treatment in which seeds are immersed in a solution of the root growth promoter of the present invention for a certain period of time, a film coating treatment, and a pellet coating treatment.

Examples of the treatment of seedlings in the method of promoting root growth of plants according to the present invention include a spraying treatment in which a suspension of the root growth promoter of the present invention is atomized and sprayed over surfaces of seedlings, an smearing treatment in which a wettable powder, an emulsifiable concentrate or a flowable agent of the root growth promoter of the present invention is applied to seedlings with a small amount of water added or without dilution, an immersing treatment in which seedlings are immersed in a solution of the root growth promoter of the present invention for a certain period of time.

When a plant or a growing site of plants is treated with the present compound, the amount of the present compound used for the treatment may be changed depending on the kind of the plant to be treated, formulation form, treatment period, climate condition and so on, but the effective amount of the present compound per 1,000 m² is typically within a range from 0.1 to 1,000 g, and preferably from 10 to 500 g.

The emulsifiable concentrate, wettable powder, flowable agent and microcapsule are typically diluted with water, and then sprinkled for the treatment. In these cases, the concentration of the present compound is typically within a range from 1 to 10,000 ppm, and preferably from 10 to 500 ppm. The dust formulation and granule are typically used for the treatment without being diluted.

In the treatment of seeds, the amount of the present compound per one seed is typically within a range from 0.01 to 10 mg, and preferably 0.1 to 5 mg. The amount of the present compound per 100 kg of seeds is typically within a range from 1 to 300 g, and preferably from 5 to 100 g.

In the treatment of seedlings, the amount of the present compound per one seedling is typically within a range from 0.1 to 50 mg, and preferably from 1 to 20 mg. In the case of the treatment of cutting, the amount of the present compound per one cutting is typically within a range from 0.1 to 20 mg, and preferably from 1 to 10 mg. In the treatment of the soil before or after sowing seedlings, the amount of the present compound per 1,000 m² is typically within a range from 0.1 to 100 g, and preferably from 1 to 50 g.

In the treatment of the culture solution, the concentration of the present compound in the culture solution is within a range from 0.1 to 1000 ppm, and preferably from 1 to 100 ppm.

The root growth promoter of the present invention can be used in agricultural lands such as fields, paddy fields, lawns and orchards or in non-agricultural lands.

The present invention can be used in agricultural lands for cultivating the following “plant” and so on to promote root growth of the plants and so on.

Examples of the crops are as follows:

crops: corn, rice, wheat, barley, rye, oat, sorghum, cotton, soybean, peanut, buckwheat, beet, rapeseed, sunflower, sugar cane, tobacco, etc.;

vegetables: solanaceous vegetables (eggplant, tomato, pimento, pepper, potato, etc.), cucurbitaceous vegetables (cucumber, pumpkin, zucchini, water melon, melon, squash, etc.), cruciferous vegetables (Japanese radish, white turnip, horseradish, kohlrabi, Chinese cabbage, cabbage, leaf mustard, broccoli, cauliflower, etc.), asteraceous vegetables (burdock, crown daisy, artichoke, lettuce, etc.), liliaceous vegetables (green onion, onion, garlic, and asparagus), ammiaceous vegetables (carrot, parsley, celery, parsnip, etc.), chenopodiaceous vegetables (spinach, Swiss chard, etc.), lamiaceous vegetables (Perilla frutescens, mint, basil, etc.), strawberry, sweet potato, Dioscorea japonica, colocasia, etc.,

flowers,

foliage plants,

turf grasses,

fruits: pomaceous fruits (apple, pear, Japanese pear, Chinese quince, quince, etc.), stone fleshy fruits (peach, plum, nectarine, Prunus mume, cherry fruit, apricot, prune, etc.), citrus fruits (Citrus unshiu, orange, lemon, rime, grapefruit, etc.), nuts (chestnuts, walnuts, hazelnuts, almond, pistachio, cashew nuts, macadamia nuts, etc.), berries (blueberry, cranberry, blackberry, raspberry, etc.), grape, kaki fruit, olive, Japanese plum, banana, coffee, date palm, coconuts, etc.,

trees other than fruit trees; tea, mulberry, flowering plant, roadside trees (ash, birch, dogwood, Eucalyptus, Ginkgo biloba, lilac, maple, Quercus, poplar, Judas tree, Liquidambar formosana, plane tree, zelkova, Japanese arborvitae, fir wood, hemlock, juniper, Pinus, Picea, and Taxus cuspidate), etc.

The aforementioned “plants” include plants, to which tolerance to HPPD inhibitors such as isoxaflutole, ALS inhibitors such as imazethapyr and thifensulfuron-methyl, EPSP synthetase inhibitors such as glyphosate, glutamine synthetase inhibitors such as glufosinate, acetyl-CoA carboxylase inhibitors such as sethoxydim, and herbicides such as bromoxynil, dicamba and 2,4-D has been conferred by a classical breeding method or by genetic engineering techniques.

Examples of a “plant” on which tolerance has been conferred by a classical breeding method include rape, wheat, sunflower and rice tolerant to imidazolinone ALS inhibitory herbicides such as imazethapyr, which are already commercially available under a product name of Clearfield (registered trademark). Similarly, there is a soybean on which tolerance to sulfonylurea ALS inhibitory herbicides such as thifensulfuron-methyl has been conferred by a classical breeding method, which is already commercially available under a product name of STS soybean.

Examples of a plant on which tolerance to acetyl-CoA carboxylase inhibitors such as trione oxime or aryloxy phenoxypropionic acid herbicides has been conferred by a classical breeding method include SR corn. The plant on which tolerance to acetyl-CoA carboxylase inhibitors has been conferred is described in Proceedings of the National Academy of Sciences of the United States of America (Proc. Natl. Acad. Sci. USA), vol. 87, pp. 7175-7179 (1990). A variation of acetyl-CoA carboxylase tolerant to an acetyl-CoA carboxylase inhibitor is reported in Weed Science, vol. 53, pp. 728-746 (2005) and a plant tolerant to acetyl-CoA carboxylase inhibitors can be generated by introducing a gene of such an acetyl-CoA carboxylase variation into a plant by genetically engineering technology, or by introducing a variation conferring tolerance into a plant acetyl-CoA carboxylase.

Plants tolerant to acetyl-CoA carboxylase inhibitors or ALS inhibitors or the like can be generated by introducing into the plant cell a nucleic acid for introduction of base-substitution variation represented by Chimeraplasty Technique (Gura T. 1999. Repairing the Genome's Spelling Mistakes. Science 285: 316-318) to introduce a site-directed amino acid substitution variation into an acetyl-CoA carboxylase gene or an ALS gene of the plant.

Examples of a plant on which tolerance has been conferred by genetic engineering technology include corn, soybean, cotton, rape and sugar beet which are tolerant to glyphosate, and which have been commercially available under a product name of RoundupReady (registered trademark), AgrisureGT, and so on. There are corn, soybean, cotton and rape which are made tolerant to glufosinate by genetic engineering technology, which have been commercially available under a product name of LibertyLink (registered trademark). A cotton made tolerant to bromoxynil by genetic engineering technology has been commercially available under a product name of BXN.

The aforementioned “plants” include crops genetically engineered to be able to synthesize selective toxins as known in genus Bacillus.

Examples of toxins expressed in such genetically engineered crops include: insecticidal proteins derived from Bacillus cereus or Bacillus popilliae; δ-endotoxins derived from Bacillus thuringiensis such as Cry1Ab, Cry1Ac, Cry1F, Cry1Fa2, Cry2Ab, Cry3A, Cry3Bb1 or Cry9C; insecticidal proteins such as VIP1, VIP2, VIP3 or VIP3A; insecticidal proteins derived from nematodes; toxins generated by animals, such as scorpion toxin, spider toxin, bee toxin, or insect-specific neurotoxins; mold fungi toxins; plant lectin; agglutinin; protease inhibitors such as a trypsin inhibitor, a serine protease inhibitor, patatin, cystatin, or a papain inhibitor; ribosome-inactivating proteins (RIP) such as lycine, corn-RIP, abrin, luffin, saporin, or briodin; steroid-metabolizing enzymes such as 3-hydroxysteroid oxidase, ecdysteroid-UDP-glucosyl transferase, or cholesterol oxidase; an ecdysone inhibitor; HMG-COA reductase; ion channel inhibitors such as a sodium channel inhibitor or calcium channel inhibitor; juvenile hormone esterase; a diuretic hormone receptor; stilbene synthase; bibenzyl synthase; chitinase; and glucanase.

Toxins expressed in such genetically engineered crops also include: hybrid toxins of δ-endotoxin proteins such as Cry1Ab, Cry1Ac, Cry1F, Cry1Fa2, Cry2Ab, Cry3A, Cry3Bb1, Cry9C, Cry34Ab or Cry35Ab and insecticidal proteins such as VIP1, VIP2, VIP3 or VIP3A; partially deleted toxins; and modified toxins. Such hybrid toxins are produced from a new combination of the different domains of such proteins, by using a genetic engineering technique. As a partially deleted toxin, Cry1Ab comprising a deletion of a portion of an amino acid sequence has been known. A modified toxin is produced by substitution of one or multiple amino acids of natural toxins.

Examples of such toxins and genetically engineered plants capable of synthesizing such toxins are described in EP-A-0 374 753, WO 93/07278, WO 95/34656, EP-A-0 427 529, EP-A-451 878, WO 03/052073, and so on.

Toxins contained in such genetically engineered plants are able to confer resistance particularly to insect pests belonging to Coleoptera, Hemiptera, Diptera, Lepidoptera and Nematodes, to the plants.

Genetically engineered plants, which comprise one or multiple insecticidal pest-resistant genes and which express one or multiple toxins, have already been known, and some of such genetically engineered plants have already been on the market. Examples of such genetically engineered plants include YieldGard (registered trademark) (a corn variety for expressing CrylAb toxin), YieldGard Rootworm (registered trademark) (a corn variety for expressing Cry3Bb1 toxin), YieldGard Plus (registered trademark) (a corn variety for expressing Cry1Ab and Cry3Bb1 toxins), Herculex I (registered trademark) (a corn variety for expressing Cry1Fa2 toxin and phosphinotricine N-acetyl transferase (PAT) so as to confer tolerance to glufosinate), NuCOTN33B (registered trademark) (a cotton variety for expressing Cry1Ac toxin), Bollgard I (registered trademark) (a cotton variety for expressing Cry1Ac toxin), Bollgard II (registered trademark) (a cotton variety for expressing Cry1Ac and Cry2Ab toxins), VIPCOT (registered trademark) (a cotton variety for expressing VIP toxin), NewLeaf (registered trademark) (a potato variety for expressing Cry3A toxin), NatureGard (registered trademark) Agrisure (registered trademark) GT Advantage (GA21 glyphosate-tolerant trait), Agrisure (registered trademark) CB Advantage (Btll corn borer (CB) trait), and Protecta (registered trademark).

The aforementioned “plants” also include crops produced by using a genetic engineering technique, which have ability to generate antipathogenic substances having selective action.

A PR protein and the like have been known as such antipathogenic substances (PRPs, EP-A-0 392 225). Such antipathogenic substances and genetically engineered crops that generate them are described in EP-A-0 392 225, WO 95/33818, EP-A-0 353 191, etc.

Examples of such antipathogenic substances expressed in genetically engineered crops include: ion channel inhibitors such as a sodium channel inhibitor or a calcium channel inhibitor, among which KP1, KP4 and KP6 toxins produced by viruses have been known; stilbene synthase; bibenzyl synthase; chitinase; glucanase; a PR protein; and antipathogenic substances generated by microorganisms, such as a peptide antibiotic, an antibiotic having a hetero ring and a protein factor associated with resistance to plant diseases (which is called a plant disease-resistant gene and is described in WO 03/000906). These antipathogenic substances and genetically engineered plants producing such substances are described in EP-A-0392225, WO95/33818, EP-A-0353191, and so on.

The “plant” mentioned above includes plants on which advantageous characters such as characters improved in oil stuff ingredients or characters having reinforced amino acid content have been conferred by genetically engineering technology. Examples thereof include VISTIVE (registered trademark) low linolenic soybean having reduced linolenic content) or high-lysine (high-oil) corn (corn with increased lysine or oil content).

Stack varieties are also included in which a plurality of advantageous characters such as the classic herbicide characters mentioned above or herbicide tolerance genes, harmful insect resistance genes, antipathogenic substance producing genes, characters improved in oil stuff ingredients or characters having reinforced amino acid content are combined.

### EXAMPLES

The present invention will be described in more detail by way of Formulation Examples, Seed Treatment Examples and Test Examples, but the present invention is not limited only to the following Examples. In the following Examples, the part represents part by weight unless otherwise specified.

### Formulation Example 1

Ten (10) parts of each of the compounds 1 to 26 shown in Table 1 is dissolved in a mixture of 35 parts of xylene and 35 partsof N,N-dimethylformamide, to which 14 parts of polyoxyethylene styryl phenyl ether and 6 parts of calcium dodecylbenzene sulfonate are added, and the mixture is well stirred to give a 10% emulsifiable concentrate of each compound.

### Formulation Example 2

Twenty (20) parts of each of the compounds 1 to 26 shown in Table 1 is added to a mixture of 4 parts of sodium lauryl sulfate, 2 parts of calcium lignin sulfonate, 20 parts of a synthetic hydrated silicon oxide fine powder and 54 parts of diatomaceous earth, and the mixture is stirred with a mixer to give a 20% wettable powder of each compound.

### Formulation Example 3

To 2 parts of each of the compounds 1 to 26 shown in Table 1, 1 part of a synthetic hydrated silicon oxide fine powder, 2 parts of calcium lignin sulfonate, 30 parts of bentonite and 65 parts of kaolin clay are added, followed by well mixing with stirring. Then, a suitable amount of water is added to the mixture, which is further stirred, granulated with a granulator and then air-dried to give a 2% granule of each compound.

### Formulation Example 4

One (1) part of each of the compounds 1 to 26 shown in Table 1 is dissolved in a proper amount of acetone, to which 5 parts of a synthetic hydrated silicon oxide fine powder, 0.3 parts of PAP and 93.7 parts of Fubasami clay are added, followed by well mixing with stirring. The removal of acetone by evaporation gives a 1% dust of each compound.

### Formulation Example 5

Ten (10) parts of each of the compounds 1 to 26 shown in Table 1, 35 parts of white carbon containing 50 parts of a polyoxyethylene alkyl ether sulfate ammonium salt, and 55 parts of water are mixed and the mixture is finely ground by a wet grinding method to give a 10% flowable formulation of each compound.

### Formulation Example 6

Each of the compounds 1 to 26 (0.1 parts) shown in Table 1 is dissolved in 5 parts of xylene and 5 parts of trichloroethane, followed by mixing with 89.9 parts of a deodorized kerosine to give a 0.1% oil agent.

### Seed Treatment Example 1

To 25 parts of each of the compounds 1 to 26 shown in Table 1, 65 parts of cyclohexanone, 5 parts of NINATE 401-A and 5 parts of BLAUNON BR-450 are added, followed by well mixing with stirring to give a 25% emulsifiable concentrate of each compound.

Then, the emulsifiable concentrate is diluted with water by 1,000 times to prepare a dilution, and rice seeds are soaked in the dilution for 24 hours until active ingredients are absorbed into the rice seeds to give treated seeds.

### Seed Treatment Example 2

Twenty-five (25) parts of each of the compounds 1 to 26 shown in Table 1, 25 parts of clay for the formulation, 25 parts of polyvinyl alcohol containing 50 parts of SOLGEN TW-20, and 25 parts of water are well mixed with stirring to give a material for forming pellets.

Then, cabbage seeds are embedded in the center of 20 mg of the material for forming pellets, followed by forming into spheres and further drying to give treated seeds.

### Seed Treatment Example 3

Twenty-five (25) parts of each of the compounds 1 to 26 shown in Table 1, 20 parts of white carbon containing 50% (weight) of a polyoxyethylene alkyl ether sulfate ammonium salt, and 55 parts of water are mixed and finely ground by a wet grinding method to give a 25% flowable formulation of each compound.

Cotton seeds are put in a stainless steel pot (having a volume of about 1,200 mL) equipped with a lifting blade for lifting seeds when the pot is rotated, and then the pot is inclined at an angle of about 45 degrees and mechanically rotated so that satisfactory mixing and tumbling granulating effect can be obtained in the pot.

The flowable formulation is diluted with water by 100 times and a hand sprayer is turned toward the inside of the pot, and then the dilution is directly sprayed to the center of a tumbling granulating layer of cotton seeds. Furthermore, the sprayer is stopped and low-pressure air is sprayed to seeds, and then the seed coating is immediately dried.

Thereafter, spraying using a hand sprayer is restarted. This spraying and drying cycle is repeated until a predetermined amount of a fluid suspension is applied to seeds, to give treated seeds.

### Test Example

One (1) part of each of the compound 1 and the compound 6 was dissolved in 99 parts of dimethyl sulfoxide and each of the resultant solutions was diluted with ion-exchanged water so as to adjust the concentration of each of the active ingredients to 10 ppm to prepare respective test chemical solutions. A cardboard in a seed growing bag (measuring 177 mm×163 mm, manufactured by Daiki Rika Kogyo Co., Ltd.) was impregnated with 17 mL of each of these test chemical solutions and 3 seeds of Raphanus sativus were sown on the cardboard. The bag was put in a plastic container and the plastic container was sealed. After culturing in a bright place at 25° C. for 7 days, the length of main root was measured. An average of three repetitions was determined and a root growth rate was calculated by the following equation.

Root growth rate (%)=(Average main root length of chemical substance treated group)/(Average main root length of non-treated control group)×100

As a result, the root growth rate of each compouhd is as shown in Table 2 below.

TABLE 2

| Compounds | Root growth rate (%) |
|---|---|
| Compound 1 | 155 |
| Compound 6 | 153 |

### INDUSTRIAL APPLICABILITY

According to the present invention, a root growth promoter having excellent plant root growth-promoting effect, and a method of promoting root growth of plants can be provided.

1. A root growth promoter comprising, as an active ingredient, a compound of formula (1): 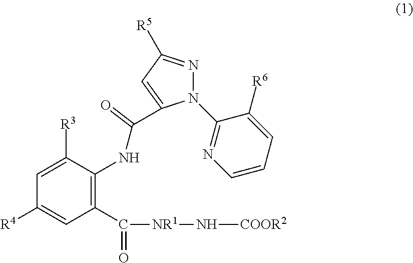
wherein R¹ represents a C1-C6 alkyl group optionally substituted with (a) halogen atom(s); R² represents a C1-C6 alkyl group optionally substituted with (a) halogen atom(s), a C3-C6 alkoxyalkyl group optionally substituted with (a) halogen atom(s), a C3-C6 alkenyl group optionally substituted with (a) halogen atom(s), or a C3-C6 alkynyl group optionally substituted with (a) halogen atom(s); R³ represents a halogen atom, or a C1-C6 alkyl group optionally substituted with (a) halogen atom(s); R⁴ represents a hydrogen atom, a halogen atom, a cyano group, or a C1-C6 alkyl group optionally substituted with (a) halogen atom(s); R⁵ represents a hydrogen atom, a halogen atom, a cyano group, a C1-C6 alkyl group optionally substituted with (a) halogen atom(s), a C1-C6 alkoxy group optionally substituted with (a) halogen atom(s), a C1-C6 alkylthio group optionally substituted with (a) halogen atom(s), a C1-C6 alkylsulfinyl group optionally substituted with (a) halogen atom(s), or a C1-C6 alkylsulfonyl group optionally substituted with (a) halogen atom(s); and R⁶ represents a halogen atom, or a C1-C6 alkyl group optionally substituted with (a) halogen atom(s).

2. A method of promoting root growth of plants, which comprises applying an effective amount of the compound of formula (1) of claim 1 to a plant or growing site of plant.

3. The method of promoting root growth of plants according to claim 2, wherein the plant is seed or seedling.

4. The method of promoting root growth of plants according to claim 2, wherein the growing site of plant is soil before or after sowing plant.

5. The method of promoting root growth of plants according to claim 2, wherein the growing site of plant is culture solution.

6. Use of the compound of formula (1) of claim 1 for promoting root growth of plants.
